(19) United States
(12) Patent Application Publication
(10) Pub. No.: US 2009/0062713 A1
(43) Pub. Date: Mar. 5, 2009

(54) BANDAGE WITH WATER SOLUBLE ADHESIVE AND METHOD OF USING SAME

(76) Inventor: Alan E. Nash, Gastonia, NC (US)

Correspondence Address: ADAMS INTELLECTUAL PROPERTY LAW, P.A., Suite 2350 Charlotte Plaza, 201 South College Street, CHARLOTTE, NC, 28244, US

(21) Appl. No.: 11/846,260

(22) Filed: Aug. 28, 2007

(51) Int. Cl. A61F 13/02; A61F 15/00

(52) U.S. Cl. 602/54; 206/441

(57) ABSTRACT

A bandage includes a wound treating section for collecting exudate from a wound, a hydrophobic layer attached to the wound treating section, and a liquid soluble adhesive positioned on the hydrophobic layer for removably adhering the bandage to a body part proximate the wound. The adhesive is dissolved upon contact with a liquid so that the bandage separates from the body part without painful sticking to the body part. The wound treating section can include a hydrocolloid or gauze pad.

### BACKGROUND AND TECHNICAL FIELD OF THE INVENTION

The invention relates to bandages. One embodiment of the invention includes a water soluble adhesive, and is particularly suited for treating slow healing and open wounds.

The treatment of slow-healing superficial wounds often requires a bandage to be placed over the wound and maintained there for a relatively long period of time. Self adhering bandages typically utilize an adhesive, that when left on the body for a long period of time, is difficult and painful to remove. As such, a bandage that sufficiently adheres to the body and yet can be easily removed when the wound is healed and/or when replacement is necessary or would be desirable.

### BRIEF SUMMARY OF THE INVENTION

Therefore, it is an object of the invention to provide a bandage that securely adheres to the body, and can be easily removed without painful sticking when desired by the user.

It is another object of the invention to provide a method for treating a slow healing wound.

These and other objects are achieved in the preferred embodiments of the invention described below. One embodiment of the invention includes a bandage having a wound treating section for collecting exudate from a wound, a hydrophobic layer attached to the wound treating section, and a liquid soluble adhesive positioned on the hydrophobic layer for removably adhering the bandage to a body part proximate the wound. The adhesive is dissolved upon contact with a liquid so that the bandage separates from the body part without painful sticking to the body part.

According to another preferred embodiment of the invention, the adhesive is water soluble.

According to another preferred embodiment of the invention, the wound treating section comprises a hydrocolloid.

According to another preferred embodiment of the invention, the wound treating section comprises gauze.

According to another preferred embodiment of the invention, the hydrophobic layer is adhered to the gauze by an insoluble adhesive.

According to another preferred embodiment of the invention, the hydrophobic layer comprises polyvinyl chloride.

According to another preferred embodiment of the invention, the bandage includes a hydrophilic layer adhered to the hydrophobic layer by the liquid soluble adhesive.

According to another preferred embodiment of the invention, the hydrophilic layer comprises nylon 66.

According to another preferred embodiment of the invention, the hydrophobic layer includes a first surface and a second surface, and the wound treating section is positioned proximate the first surface and the hydrophilic layer is positioned proximate the second surface.

According to another preferred embodiment of the invention, the bandage is positioned on a body part, the wound treating section is proximate the body part and the hydrophilic layer is distal from the body part and forms a top cover of the bandage so that liquid introduced on the hydrophilic layer is allowed to seep through to the liquid soluble adhesive.

According to another preferred embodiment of the invention, the bandage includes first and second liner sections removably adhered to the wound treating section.

According to another preferred embodiment of the invention, a net overlaid the wound treating section.

According to another preferred embodiment of the invention, first and second liner sections are removably adhered to the net.

According to another preferred embodiment of the invention, a bandage kit includes a bandage having a wound treating section for collecting exudate from a wound, a hydrophobic layer attached to the wound treating section, a liquid soluble adhesive positioned on the hydrophobic layer for removably adhering the bandage to a body part proximate the wound, and a hydrophilic cover layer positioned on the liquid soluble adhesive. A spraying device containing a liquid solution adapted for dissolving the liquid soluble adhesive is provided. The spraying is device adapted for delivering a fine mist of the liquid solution that permeates the hydrophilic cover layer and dissolves the liquid soluble adhesive.

According to another preferred embodiment of the invention, the liquid soluble adhesive is water soluble, and the liquid solution comprises sterile water, aloe vera solids, sodium benzoate and potassium sorbate.

According to another preferred embodiment of the invention, the spraying device comprises a bottle containing approximately twenty milliliters of the liquid solution, and a 130 microliter pump.

According to another preferred embodiment of the invention, an envelope package is provided for storing the bandage until it is to be used.

According to another preferred embodiment of the invention, a method for treating a wound includes the steps of providing a bandage having a wound treating section for collecting exudate from a wound, a hydrophobic layer attached to the wound treating section, a liquid soluble adhesive positioned on the hydrophobic layer for removably adhering the bandage to a body part proximate the wound, and a hydrophilic cover layer positioned on the liquid soluble adhesive. The bandage is positioned on the user's body part to cover the wound, and a fine mist of liquid solution adapted for dissolving the liquid soluble adhesive is sprayed onto the hydrophilic cover layer so that the liquid solution permeates the hydrophilic cover layer and dissolves the liquid soluble adhesive.

### BRIEF DESCRIPTION OF THE DRAWINGS

The invention may be best understood by reference to the following description taken in conjunction with the accompanying drawing figures in which:

FIG. 1 is a perspective view of a body part with a wound to be treated;

FIG. 2 is an environmental perspective view of a bandage according to a preferred embodiment of the invention;

FIG. 3 is a perspective view illustrating a preferred method of using the bandage of FIG. 2 according to a preferred embodiment of the invention;

FIG. 4 is another perspective view illustrating the method of FIG. 3;

FIG. 5 is a cross sectional view of the bandage of FIG. 2;

FIG. 6 is a perspective view of the bandage of FIG. 2 within an envelope package; and

FIG. 7 is a cross sectional view of a bandage according to another preferred embodiment of the invention.

### DETAILED DESCRIPTION OF THE INVENTION

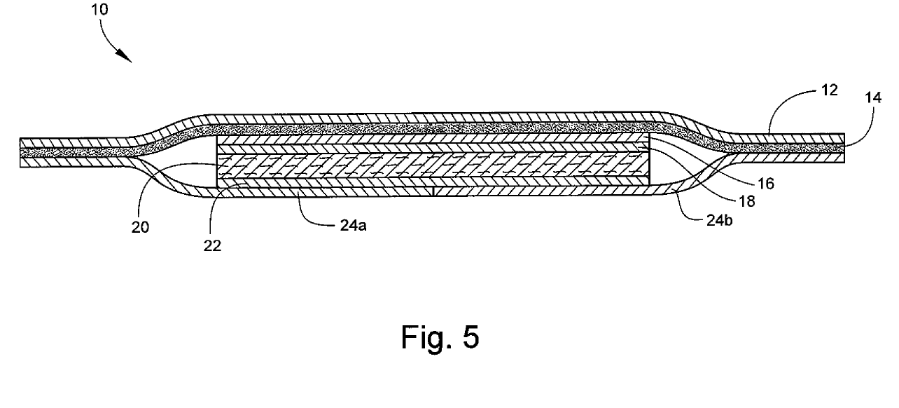

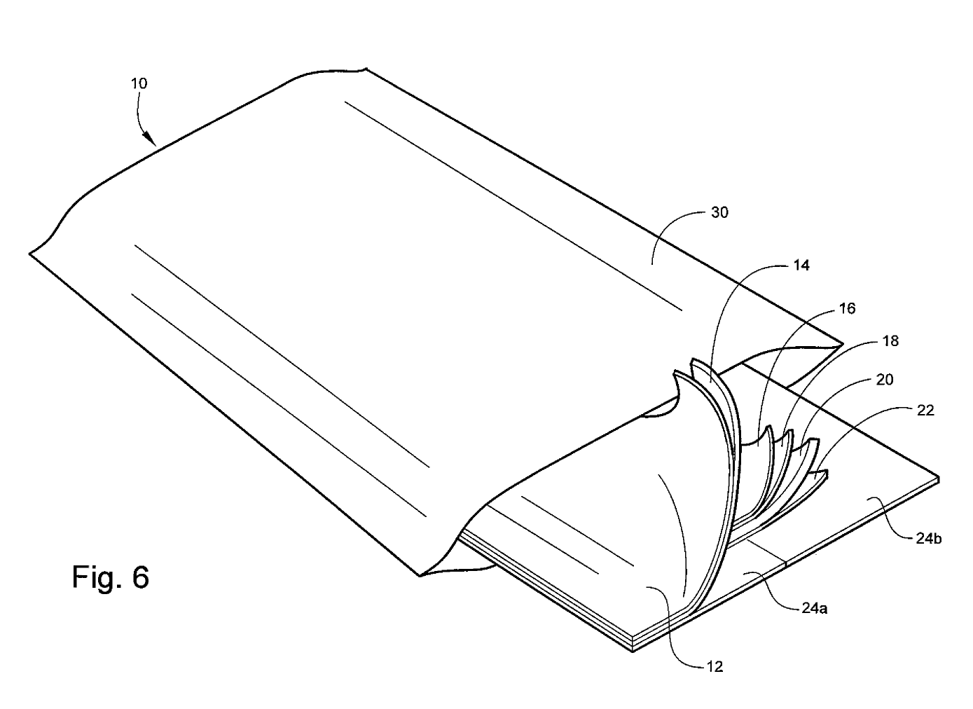

Referring now to the drawings, a bandage according to a preferred embodiment of the present invention is illustrated in FIGS. 2-6, and shown generally at reference numeral 10. As shown in FIG. 5, the bandage 10 includes a top cover layer 12 having a hydrophilic material such as nylon 66, a water soluble adhesive 14 underneath the hydrophilic cover layer 12, a hydrophobic layer 16 below the water soluble adhesive 14, and a wound treating section, such as a gauze pad 20 underneath the hydrophobic layer 16.

The hydrophobic layer 16 can be a polyvinyl chloride film, and is adhered to the hydrophilic cover layer 12 by the water soluble adhesive 14. The hydrophobic layer 16 is adhered to the gauze pad 20 by a layer of water insoluble adhesive 18.

Netting 22 is overlaid onto the gauze pad 20 to hold the gauze 20 in place and minimize separation of filaments from the rest of the gauze 20. This helps prevent gauze filaments from remaining in a wound after removal of the bandage 10. The netting 22 can be comprised of a non-stick woven material.

A bottom layer of the bandage 10 has two overlapping liner segments 24a, 24b that are removably adhered to the netting 22 and gauze 20. The liner segments 24a, 24b cover the gauze 20 and netting 22, and keep them clean and unexposed to the elements until the bandage 10 is to be used. As shown in FIG. 5, the opposing ends of the liner segments 24a, 24b are adhered to the ends of the hydrophilic layer 12 by the water soluble adhesive 14 to seal and enclose the contents of the bandage 10 together.

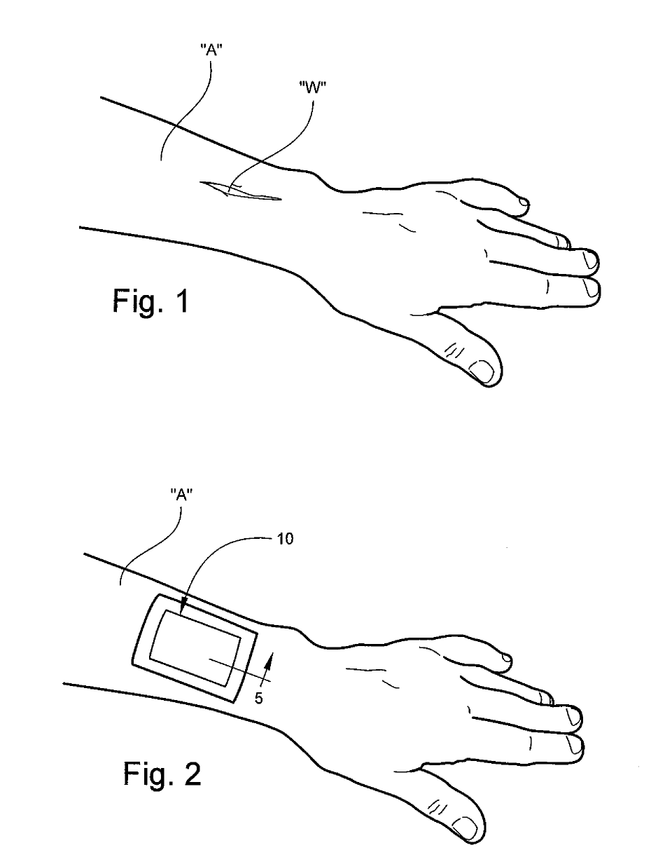

FIGS. 1-4 illustrate a method of using the bandage 10 according to a preferred embodiment of the invention. FIG. 1 shows a wound “W” on a body part of the user such as an arm “A”. The liner segments 24a, 24b are sized and positioned to be slightly overlapping at the center junction proximate the mid-point of the gauze 20 and netting 22. As such, when the bandage is ready for use, the user peels back the two liner segments 24a, 24b to expose the gauze 20 and portions of the water soluble adhesive 14 for adhering on a body part of the user. The bandage 10 is positioned on the user's body such that the gauze 20 and netting 22 cover the wound “W”, as shown in FIG. 2.

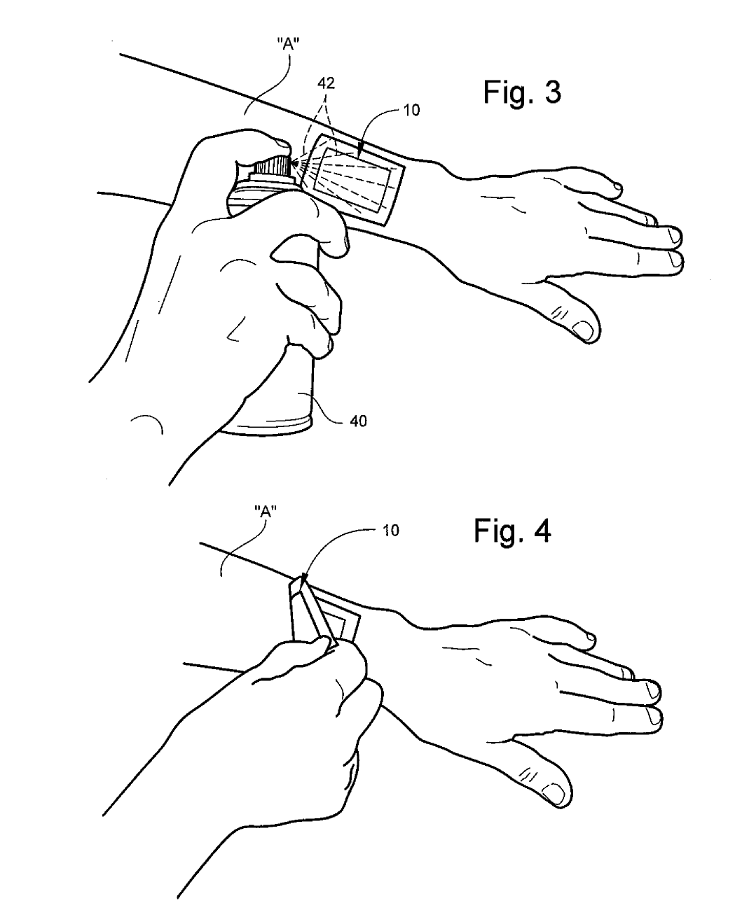

As shown in FIG. 3, a spray bottle 40 is provided containing a liquid solution that is formulated for dissolving the water soluble adhesive 14. When the user desires to remove the bandage 10, a fine mist 42 of the liquid solution is sprayed onto the bandage 10, as shown in FIG. 3. The mist 42 of liquid solution permeates the hydrophilic cover layer 12 and seeps into the water soluble adhesive 14. The liquid solution dissolves the adhesive 14, making it easy for the user to remove the bandage 10 without painful sticking to the user's body, as shown in FIG. 4.

Preferably, the liquid solution is 78.6% sterile water, 20% aloe vera solids, 0.2% sodium benzoate and 0.2% potassium sorbate. The spray bottle 40 preferably holds 20 milliliters and includes a 130 microliter pump.

As shown in FIG. 6, the bandage 10 can be stored within a package 30 until ready for use. The package 30 preferably includes two polyester sheets heat sealed to form an envelope.

One preferred embodiment of the bandage 10 preferably has the following dimensions: the hydrophilic cover layer 12 and the water soluble adhesive 14 are each 4×3 inches; the hydrophobic film 16, the insoluble adhesive 18, and the gauze 20 and the net 22 are each 3×2 inches; each of the liner segments 24a, 24b is 4×1⅝ inches; and each sheet of the envelope package 30 is 4½×3½ inches.

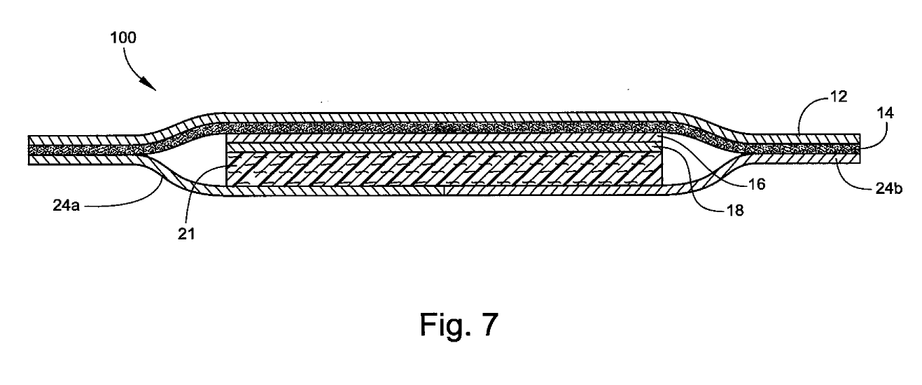

A bandage according to another preferred embodiment of the invention is illustrated in FIG. 7, and shown generally at reference numeral 100. The bandage 100 is similar in structure the previously described bandage 10, except that it utilizes a hydrocolloid 21, rather than the gauze pad 20 of bandage 10.

A bandage, a bandage kit, and a method of using same are described above. While specific embodiments of the present invention have been described, it will be apparent to those skilled in the art that various modifications thereto can be made without departing from the spirit and scope of the invention. Accordingly, the foregoing description of the preferred embodiments of the invention and the best mode for practicing the invention are provided for the purpose of illustration only and not for the purpose of limitation, the invention being defined by the claims.

What is claimed is:

1. A bandage comprising:
(a) a wound treating section for collecting exudate from a wound;
(b) a hydrophobic layer attached to the wound treating section; and
(c) a liquid soluble adhesive positioned on the hydrophobic layer for removably adhering the bandage to a body part proximate the wound, while permitting the adhesive to dissolve upon contact with a liquid and separates the bandage from the body part.

2. A bandage according to claim 1, wherein the adhesive is water soluble.

3. A bandage according to claim 1, wherein the wound treating section comprises a hydrocolloid.

4. A bandage according to claim 1, wherein the wound treating section comprises gauze.

5. A bandage according to claim 4, wherein the hydrophobic layer is adhered to the gauze by an insoluble adhesive.

6. A bandage according to claim 1, wherein the hydrophobic layer comprises polyvinyl chloride.

7. A bandage according to claim 1, further comprising a hydrophilic layer adhered to the hydrophobic layer by the liquid soluble adhesive.

8. A bandage according to claim 7, wherein the hydrophilic layer comprises nylon 66.

9. A bandage according to claim 7, wherein the hydrophobic layer includes a first surface and a second surface, and the wound treating section is positioned proximate the first surface and the hydrophilic layer is positioned proximate the second surface.

10. A bandage according to claim 9, wherein when the bandage is positioned on a body part, the wound treating section is proximate the body part and the hydrophilic layer is distal from the body part and forms a top cover of the bandage for allowing liquid introduced on the hydrophilic layer to seep through to the liquid soluble adhesive.

11. A bandage according to claim 1, further comprising first and second liner sections removably adhered to the wound treating section

12. A bandage according to claim 1, further comprising a net overlaid the wound treating section.

13. A bandage according to claim 12, further comprising first and second liner sections removably adhered to the net.

14. A bandage kit comprising:
(a) a bandage comprising:
(i) a wound treating section for collecting exudate from a wound,
(ii) a hydrophobic layer attached to the wound treating section,
(iii) a liquid soluble adhesive positioned on the hydrophobic layer for removably adhering the bandage to a body part proximate the wound, and
(iv) a hydrophilic cover layer positioned on the liquid soluble adhesive; and
(b) a spraying device containing a liquid solution adapted for dissolving the liquid soluble adhesive, the spraying device adapted for delivering a fine mist of the liquid solution that permeates the hydrophilic cover layer and dissolves the liquid soluble adhesive.

15. A bandage kit according to claim 14, wherein the wound treating section comprises a hydrocolloid.

16. A bandage kit according to claim 14, wherein the wound treating section comprises gauze.

17. A bandage kit according to claim 14, wherein the hydrophobic layer comprises polyvinyl chloride.

18. A bandage kit according to claim 14, wherein the hydrophilic layer comprises nylon 66.

19. A bandage kit according to claim 14, wherein the liquid soluble adhesive is water soluble, and the liquid solution comprises sterile water and aloe vera solids.

20. A bandage kit according to claim 19, wherein the liquid solution further comprises sodium benzoate and potassium sorbate.

21. A bandage kit according to claim 14, wherein the spraying device comprises a bottle containing approximately twenty milliliters of the liquid solution, and a 130 microliter pump.

22. A bandage kit according to claim 14, further comprising an envelope package for containing the bandage.

23. A method for treating a wound, comprising the steps of:
(a) providing a bandage comprising:
(i) a wound treating section for collecting exudate from a wound,
(ii) a hydrophobic layer attached to the wound treating section,
(iii) a liquid soluble adhesive positioned on the hydrophobic layer for removably adhering the bandage to a body part proximate the wound, and
(iv) a hydrophilic cover layer positioned on the liquid soluble adhesive;
(b) positioning the bandage on the body part to cover the wound; and
(c) spraying a liquid solution onto the liquid soluble adhesive onto the hydrophilic cover layer, permeating the hydrophilic cover layer, and dissolving the liquid soluble adhesive.
